(19) United States
(12) Patent Application Publication
(10) Pub. No.: US 2025/0152805 A1
(43) Pub. Date: May 15, 2025

(54) CARTON

(71) Applicant: MM NEWPORT LTD., Bradford (GB)

(72) Inventor: Steven Gerard ARCHAMBAULT, Pemberton, NJ (US)

(21) Appl. No.: 18/838,842

(22) PCT Filed: Feb. 16, 2023
(86) PCT No.: PCT/EP2023/053948

§ 371 Date: Aug. 15, 2024

Related U.S. Application Data
(63) Continuation of application No. 17/674,294, filed on Feb. 17, 2022, now abandoned.

(51) Int. Cl. A61M 5/00; A61M 5/31

(52) U.S. Cl. CPC A61M 5/002; A61M 5/3129; A61M 2207/00

(57) ABSTRACT

The present invention relates to a carton for obscuring the contents of the barrel of a syringe, the carton comprising; a housing; wherein the housing comprises an opening through which the tip, barrel and flange of a syringe may be introduced into the housing; a chamber with a first portion for receiving the barrel of the syringe and a second portion for receiving the flange of the syringe; and an aperture opposite to the opening, through which the tip of the syringe may protrude when the syringe is introduced into the housing.

This invention relates to packaging. In particular, the invention provides a carton that obscures the contents of the barrel of a syringe.

### BACKGROUND

Blind clinical trials remove patient and/or practitioner bias and consequently, may limit the potential placebo effect. To comply with the requirements of blinded clinical trials it is essential that the recipient of a drug, and often the practitioner administering the drug, do not know whether they are receiving or administering the placebo or the drug. Trials wherein both the practitioner and the patient do not know whether they are receiving or administering the placebo or the drug are called “double-blind”.

Current drug/placebo blinding possibilities include the use of vial ampoule blinding solutions, for example opaque vials comprising drug or placebo, plastic syringe blinding systems, for example blacked-out syringe barrels or plastic syringe covers and make-shift systems, for example covering a vial or syringe with a cloth.

However, in the case of many of the above blinding possibilities, the placebo or drug may need to be packaged differently, or decanted into new blacked out or opaque packaging for the trial which may be both inconvenient and expensive for the user. Further, make shift systems may fall off during administration of the placebo or drug and accordingly, may compromise the validity of the blind clinical trial.

Thus, there is a need for an effective syringe cloaking carton for blind clinical trial testing that is user-friendly, effective, easy to manufacture and economical.

### SUMMARY OF THE INVENTION

According to the present invention there is provided a carton for obscuring the contents of the barrel of a syringe, the carton comprising; a housing; wherein the housing comprises an opening through which the tip, barrel and flange of a syringe may be introduced into the housing; a chamber with a first portion for receiving the barrel of the syringe and a second portion for receiving the flange of the syringe; and an aperture opposite to the opening, through which the tip of the syringe may protrude when the syringe is introduced into the housing.

As used herein, the term “syringe” refers to the assembled plunger, barrel, flange and tip [of a syringe], wherein the tip may comprise a needle and/or a protective cover (i.e., cap). Thus, it will be appreciated that the term “syringe” may refer to a syringe which includes a needle and/or a protective cover (i.e., cap) on the tip. It will be further appreciated that the term “syringe” refers to any syringe (including e.g., a pre-filled syringe) that is known in the art.

The carton may comprise at least one flap portion which, following introduction of the syringe (i.e., tip, barrel and flange) into the housing may be moved from a first (e.g., unlocked) configuration in which it does not engage with the syringe, into a second (e.g., locked) configuration in which it engages with the flange of the syringe to thereby secure the syringe within the housing.

Preferably, the carton comprises at least two flap portions which, following introduction of the syringe (i.e., tip, barrel and flange) into the housing may be moved from a first (e.g., unlocked) configuration in which they do not engage with the syringe into a second (e.g., locked) configuration in which they engage with the flange of the syringe to thereby secure the syringe within the housing.

Following the introduction of the syringe (i.e., tip, barrel and flange) into the housing, the barrel of the syringe is within the first portion of the chamber, the flange of the syringe is within the second portion of the chamber, the tip of the syringe protrudes through the aperture such that it is located outside of the housing and the plunger of the syringe is located outside the opening of the housing. Thus, as used herein the phrases “syringe within the housing” or “syringe in the housing” refer to the syringe in the above arrangement.

To facilitate the introduction of the tip, barrel and flange of a syringe into the housing, the flap portion or portions should be in (or moved into) a first (e.g., unlocked) configuration such that they are positioned within the opening and do not engage with the syringe. Following the introduction of the tip, barrel and flange of a syringe into the housing, and subsequent protrusion of the tip through the aperture of the housing, the flap portion(s) may be in (i.e., moved into) a second (e.g., locked) configuration in which it or they engage(s) with the flange of the syringe to thereby secure the syringe within the housing.

As used herein, the phrase “secure the syringe within the housing” means the movement of the syringe back through the opening of the housing is substantially prevented. In other words, in the second (e.g., locked) configuration the flap portion(s) impedes the withdrawal of the tip, barrel and flange of the syringe back through the opening of the housing, thereby preventing the carton from falling off the syringe during use (e.g., administration of the contents of the barrel of the syringe to a patient). The user (e.g., syringe user) may move the flap portion(s) between the first (e.g., unlocked) configuration and the second (e.g., locked) configuration following the introduction of the tip, barrel and flange of a syringe into the housing.

It will be appreciated that the flap portion or portions include(s) a first end and a second end. The first end of the or each flap portion may be attached to the housing in a position substantially adjacent to the opening of the housing. Preferably, the first end of the or each flap portion is attached to the housing on the edge or rim of the opening of the housing. Preferably, the first end of the or each flap portion is a continuation of the housing (i.e., formed of the same continuous sheet of material). Where a carton of the invention comprises at least two flap portions, preferably the flap portions are uniformly distributed on/around the edge or rim of the opening of the housing.

The opening of the housing may be substantially square or oblong. Where a carton of the invention comprises at least one or at least two flap portions, the or each flap portion may be positioned on the side of the square or oblong opening of the housing (i.e., on a side/edge of the opening). Where a carton of the invention comprises at least two flap portions, the flap portions are preferably positioned on opposing sides of the square or oblong opening of the housing (i.e., on opposite sides/edges of the opening).

Preferably, the flap portion or portions are dimensioned such that when in the second (e.g., locked) configuration, the second end of the or each flap portion abuts against the flange of the syringe to thereby secure the syringe within the housing. The flap portion or portions may provide resistance against the movement of the syringe back through the opening of the housing thereby preventing the carton from falling off the syringe during use. However, it will be appreciated that, if required (i.e., following use of the syringe), the user will be able to remove the syringe from the carton by moving the flap portion(s) between the second (e.g., locked) configuration to the first (e.g., unlocked) configuration and removing the tip, barrel and flange of a syringe out of the opening.

The second portion of the chamber may comprise indents (e.g., disposed in a position opposite to the opening) which are ergonomically designed (e.g., semi-circular) to engage with the fingers (e.g., fore finger and middle finger) of the user (e.g., syringe user). The indents may provide a rest whereby the fingers of the user can be placed during use of the syringe (e.g., administration of the contents of the barrel of the syringe to a patient). The indents may be shaped and dimensioned such that they are complementary to the fingers of the user, thereby facilitating use of the syringe.

The second portion of the chamber may comprise plunger indents (e.g., disposed in a position adjacent to the opening) which are ergonomically designed (e.g., semi-circular) to allow (e.g., full) depression of the plunger of the syringe. It will be appreciated that for the syringe to be used (e.g., the contents of the barrel of the syringe administered to a patient) the user (e.g., syringe user) presses down on the plunger of the syringe until the plunger reaches a desired position in the barrel. The plunger indents may be shaped and dimensioned such that they allow the (e.g., full) depression of the plunger of the syringe. Therefore, the complete contents of the barrel of the syringe may be administered whilst the syringe is within the housing.

Preferably, the housing is at least substantially opaque. A substantially opaque housing enables the contents of the barrel of a syringe to be obscured or partially obscured from the user (e.g., syringe user), practitioner or patient etc. following the introduction of the syringe (i.e., the tip, barrel and flange) into the housing.

As used herein the phrase obscured or partially obscured means that the user (e.g., syringe user), practitioner or patient etc. is unable to identify (e.g., by reading the label) the contents of a syringe (e.g., syringe barrel) in a carton of the invention and/or distinguish between a carton of the invention comprising a syringe with first substance therein (e.g., within the syringe barrel) and a carton of the invention comprising a syringe with a second (i.e., different) substance therein (e.g., within syringe barrel). However, will be appreciated that it may still be possible for the user (e.g., syringe user), practitioner or patient etc. to see the barrel, flange and/or label of a syringe within a carton of the invention without being able to identify or distinguish between the substances therein.

Preferably, the housing is substantially complimentary in shape (e.g., T-shaped prism) to the syringe. When the housing is substantially complimentary in shape to the syringe, the use of the syringe (e.g., administration of the contents of the barrel to a patient) within the housing is simple and may be comparable to that of a naked syringe. However, it will be appreciated that the housing may be any shape (e.g., cuboid, e.g., triangular prism).

The opening of the housing is dimensioned such that the tip, barrel and flange of a syringe may be introduced into the housing through the opening. The first portion of the chamber is dimensioned to receive the barrel of the syringe and the second portion of the chamber is dimensioned to receive the flange of the syringe. The aperture is dimensioned such that the tip of a syringe may protrude through the aperture when the syringe is introduced into the housing.

Preferably, the housing is dimensioned such that following introduction of the syringe into the housing, movement of the syringe (i.e., within the housing) is substantially prevented. Movement of the syringe within the housing may hinder use of the syringe (e.g., administration of the contents of the barrel of the syringe to a patient). Therefore, it is preferable that movement of the syringe (i.e., within the housing) is substantially prevented to facilitate use of the syringe.

The carton may be formed from the same continuous sheet of material. The continuous sheet of material may be held in shape with an adhesive (e.g., glue). The adhesive (e.g., glue) may be present on a tab portion of the continuous sheet of material. Preferably, the material used to form the carton is a paper based material e.g., paperboard, e.g., cardboard. The carton can be produced using conventional manufacturing procedures and is easy to manufacture.

The carton may have a syringe disposed therein. The syringe may be a pre-filled syringe. The syringe may contain (e.g., within the barrel) a substance. The substance may be a pharmaceutical or healthcare product. The product may be a liquid or a powder. However, it will be appreciated that a powder must be reconstituted in solution prior to administration to a patient. Preferably, the barrel of the syringe is within the first portion of the chamber, the flange of the syringe is within the second portion of the chamber, the tip of the syringe protrudes through the aperture such that it is located outside of the housing and the plunger of the syringe is located outside the opening of the housing. Preferably, the syringe is dimensioned such that following introduction of the syringe into the housing, movement of the syringe (i.e., within the housing) is substantially prevented.

In a further aspect of the present invention, there is provided a use of a carton for obscuring the contents of the barrel of a syringe. The carton may obscure the contents of the barrel of a syringe whilst being administered to a patient during clinical trial testing (e.g., Clinical Trials Phase 3 and Phase 4 studies).

In a further aspect of the present invention, there is provided a blank for forming the carton according to any statement set out herein.

In a further aspect of the present invention, there is provided a blank for a carton comprising; a single continuous sheet of material, the single sheet comprising; a first section which forms a back panel of the carton; a second section which forms a first side panel of the carton; a third section which forms a front panel of the carton; and a fourth section which forms a second side panel of the carton; wherein the first section is joined to the second section along a fold line and/or perforated crease; wherein the second section is joined to the third section along a fold line and/or perforated crease; wherein the third section is joined to the fourth section along a fold line and/or perforated crease; at least one flap extending longitudinally from one or more of the sections in substantially the same direction as at least one of the fold lines and/or perforated creases; and a tab extending longitudinally from the first section in a direction substantially perpendicular to at least one of the fold lines and/or perforated creases.

The blank may further comprise at least two cuts, the first cut traversing the second section and a portion of the third section in a direction substantially perpendicular to at least one of the fold lines and/or perforated creases and the second cut traversing the fourth section and a portion of the third section in a direction substantially perpendicular to at least one of the fold lines and/or perforated creases.

The blank may further comprise indents (e.g., cut-outs) in the first section and the third section.

The blank may be folded along the fold lines and/or perforated creases such that the tab contacts the underside of the fourth section and is attached thereto with adhesive to thereby join the tab and the fourth section together.

The blank may be folded along a further fold line and/or perforated crease such that a portion of the fourth section contacts a portion of the first section and is attached thereto with adhesive to thereby join a portion of the first section to a portion of the fourth section.

It will be appreciated that the skilled person will be able to fold the blank along the fold lines and/or perforated creases to form a carton.

In a further aspect of the present invention, there is provided a method of making a carton comprising folding a blank as set out herein. Advantageously, the carton is easy to erect from the blank.

An embodiment of the invention will now be described, by way of example only, with reference to the accompanying drawings, in which:

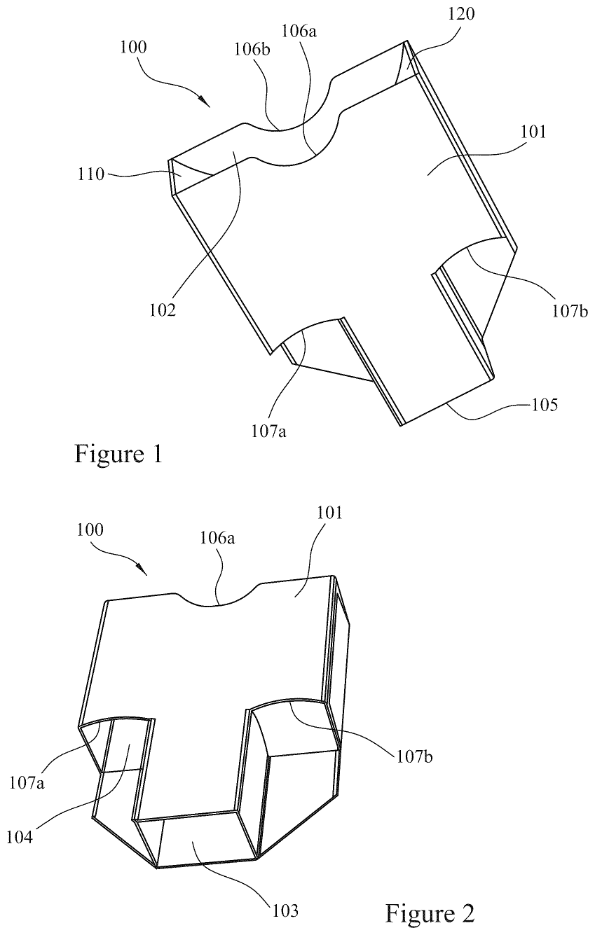

FIG. 1 is a perspective view of a carton according to the present invention;

FIG. 2 is an alternative perspective view of the carton of FIG. 1;

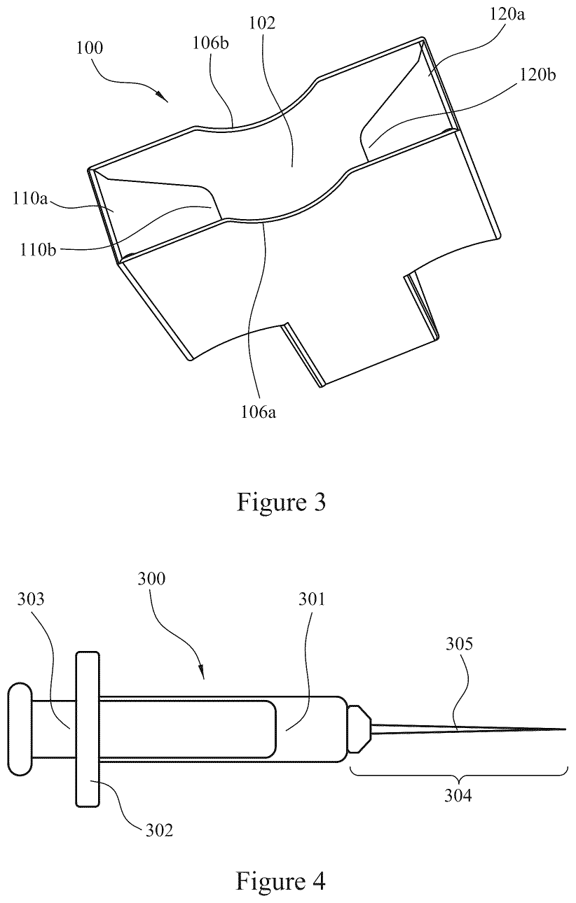

FIG. 3 is an alternative perspective view of the carton of FIG. 1;

FIG. 4 is a perspective view of an assembled syringe which may be disposed within a carton according to the present invention;

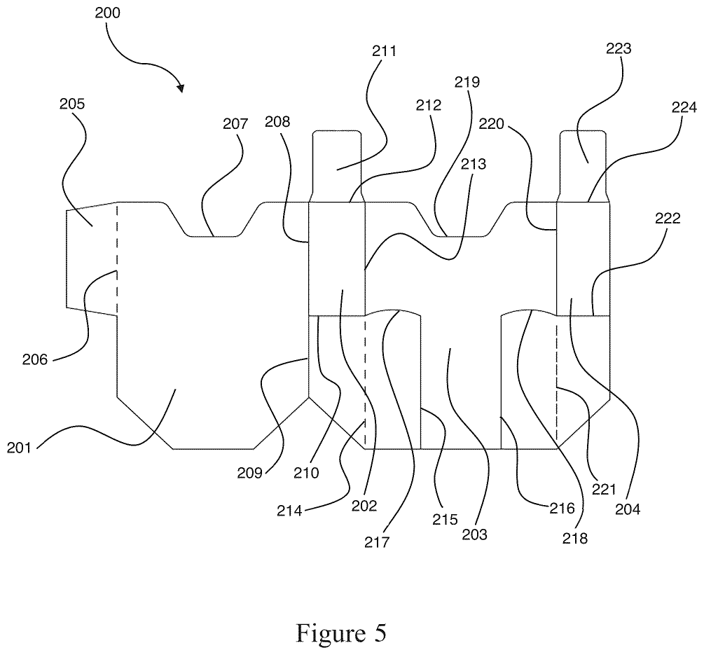

FIG. 5 is a view of a blank of the carton of FIG. 1;

FIGS. 1, 2 and 3 illustrate a carton for obscuring the contents of the barrel of a syringe according to one aspect of the present invention. FIGS. 2 and 3 illustrate a different view of the same carton as FIG. 1. FIG. 5 illustrates a blank of a carton according to an aspect of the present invention.

The carton 100 comprises a housing 101, a first flap portion 110 and a second flap portion 120 and is formed from the same continuous sheet of paper (e.g., paperboard) or other suitable material (e.g., cardboard). The continuous sheet of paper (e.g., paperboard) or other suitable material (e.g., cardboard) is substantially opaque. The carton 100 is capable of containing a syringe (including e.g., a pre-filled syringe) (see FIG. 4) which comprises a liquid pharmaceutical product therein (i.e., within the barrel), for example a drug or placebo.

The housing 101 has an oblong opening 102 at one end of the housing 101 through which the tip, barrel and flange of a syringe (i.e., an assembled syringe, see e.g., FIG. 4) may be introduced into the housing 101, a chamber with a first portion 103 for receiving the barrel of the syringe and a second portion 104 for receiving the flange of the syringe, and an aperture 105 opposite to the opening 102, through which the tip of the syringe may protrude when the syringe is introduced into the housing 101.

The second portion of the chamber 104 has semi-circular indents 107a and 107b which are disposed in the wall of the second portion of the chamber 104 which is opposite to the opening 102, and positioned on either side of the entrance to the first portion of the chamber 103. Indents 107a and 107b are complementary in shape to the fore finger and middle finger of the user and dimensioned to fit the fore finger and middle finger of the user. The second portion of the chamber 104 has semi-circular plunger indents 106a and 106b which are disposed adjacent to, and on opposing sides of the opening 102, which are dimensioned to allow full depression of the plunger of the syringe.

The opening 102 is dimensioned such that the tip, barrel and flange of a syringe may be introduced into the housing through the opening 102. The first portion of the chamber 103 is dimensioned to receive the barrel of a syringe and the second portion of the chamber 104 is dimensioned to receive the flange of the syringe. The aperture 105 is dimensioned such that the tip of a syringe (including the needle and/or protective cap) may protrude through the aperture 105 when the syringe is introduced into the housing 101.

It will be understood that the two flap portions 110 and 120 are a continuation of the housing 101, formed of the same continuous sheet of paper (e.g., paperboard) or other suitable material (e.g., cardboard). The two flap portions 110 and 120 each have a first end 110a/120a and a second end 110b/120b. The first ends 110a/120a of the two flap portions 110 and 120 are attached to the housing 101 at the edge of opening 102 on opposing sides. As shown in FIG. 1, when in the first (e.g., unlocked) configuration, the two flap portions 110 and 120 are arranged such that they are positioned within the opening 102 and do not engage with a syringe.

Following introduction of a syringe (i.e., the tip, barrel and flange) (not shown in FIG. 1) into the housing 101, the two flap portions 110 and 120 may be moved from the first (e.g., unlocked) configuration into a second (e.g., locked) configuration in which the second ends 110b/120b abut against the flange of the syringe to thereby secure the syringe within the housing 101. The two flap portions 110 and 120 are dimensioned such that when in the second (e.g., locked) configuration, the second ends 110b/120b provide resistance against the movement of the syringe back through the opening 102 of the housing 101 thereby preventing the carton 100 from falling off the syringe during use. However, it will be appreciated that, if required (i.e., following use of the syringe), the user will be able to remove the syringe from the housing 101 by moving the two flap portions 110 and 120 between the second (e.g., locked) configuration to the first (e.g., unlocked) configuration and removing the tip, barrel and flange of a syringe out of the opening 102.

FIG. 4 is a schematic view of an assembled syringe which may be disposed within a carton according to the present invention. The syringe 300 comprises a barrel 301, flange 302, plunger 303, and tip 304 with needle 305.

FIG. 5 illustrates a blank 200. The blank illustrated in FIG. 5 is configured to form the carton 100 shown in FIGS. 1, 2 and 3.

The blank 200 comprises a first section 201 which forms a back panel of the carton, a second section 202 which forms a first side panel of the carton, a third section 203 which forms a front panel of the carton and a fourth section 204 which forms a second side panel of the carton. The first section 201 is joined to the second section along fold lines 208 and 209. The second section 202 is joined to the third section 203 along fold line 213 and perforated crease 214. The third section 203 is joined to the fourth section 204 along fold line 220 and perforated crease 221.

Flap 211 extends longitudinally from the second section 202 in substantially the same direction as fold line 208 and is joined to the second section 202 along fold line 212. Flap 223 extends longitudinally from the fourth section 204 in substantially the same direction as fold line 220 and is joined to the fourth section along fold line 224.

Tab 205 extends longitudinally from the first section 201 in a direction substantially perpendicular to perforated crease 206 and is joined to first section 201 along perforated crease 206.

The first section 201 is indented to form plunger indent 207. The third section 203 is indented to form plunger indent 219, which is shaped and dimensioned identical to plunger indent 207.

The second section 202 comprises a cut 210, which traverses the second section 202 (from fold lines 209/208 to perforated crease 214/fold line 213) in a direction substantially perpendicular to fold line 209.

The third section 203 has two parallel fold lines 215, 216, which extend longitudinally of the third section 203, from the edge of third section 203 for substantially over half the length of the third section 203. Semi-circular shaped cut 217 extends from fold line 215 to the second section 202 (where perforated crease 214, cut 210 and fold line 213 converge) in a direction substantially perpendicular to fold line 215. Semi-circular shaped cut 218 extends from fold line 216 to the fourth section 204 (where perforated crease 221, cut 222 and fold line 220 converge) in a direction substantially perpendicular to fold line 216.

The fourth section 204 comprises a cut 222, which traverses the fourth section 204 (from the edge of the fourth section 204 to perforated crease 221/fold line 220) in a direction substantially perpendicular to fold line 220.

It will be appreciated that the blank 200 may be assembled by the skilled person to form the carton of FIG. 1. An example of a method for assembling the blank 200 is described below.

To assemble the blank 200 into the carton 100, the blank is folded along the fold lines and/or perforated creases such that tab 205 contacts the underside of the fourth section 204 and is attached thereto with adhesive to thereby join the tab 205 and the fourth section 205 together.

The blank 200 is then folded along a further fold line and/or perforated crease such that a portion of the fourth section 204 contacts a portion of the first section 201 and is attached thereto with adhesive to thereby join a portion of the first section 201 to a portion of the fourth section 204.

The second section 202 is then moved perpendicular to the first section 201. The third section 203 moves synchronously with the second section 202, such that a portion of the third section 203 is parallel to the first section 201 and a portion of the third section 203 (between fold line 216 and perforated crease 221) is perpendicular to the first section 201.

A portion of the second section 202 (i.e., between fold line 209 and perforated crease 214) and a portion of the third section 203 (i.e., between fold line 215 and perforated crease 214) are then folded towards the underside of the first section 201 along fold lines 215, 209 and perforated crease 214 such that the portion of the second section 202 contacts the first section 201 and the portion of the third section 203 is perpendicular to the first section 201.

Opening 102 is formed from the edges of the first section 201, second section 202, third section 203 and fourth section 204. Flap 211 and flap 223 form flap portions 110 and 120 respectively, positioned on the opposite sides of opening 102.

The first portion of the chamber 103 is formed from a portion of the underside of the first section 201, the undersides of the portions of the third section 203 which are perpendicular to the first section 201 and a portion of the third section 203 which is parallel to the first section 201. Aperture 105 is formed from a portion of the edge of first section 201, the portions of the edge of the third section 203 which are perpendicular to first section 201 and a portion of the edge of the third section 203 which is parallel to the first section 201 (between fold line 125 and fold line 216).

The second portion of the chamber 104 is formed from portions of the undersides of first section 201, second section 202, third section 203 and fourth section 204.

Semi-circular shaped cuts 217 and 218 form indents 107a and 107b which are disposed in the wall of the second portion of the chamber 104. Indents 207 and 219 form plunger indents 106a and 106b which are disposed adjacent to, and on opposing sides of the opening 102.

1. A carton for obscuring contents of a barrel of a syringe, the carton comprising:
a housing;
wherein the housing comprises an opening through which a tip, the barrel and a flange of the syringe may be introduced into the housing;
a chamber with a first portion for receiving the barrel of the syringe and a second portion for receiving the flange of the syringe; and
an aperture opposite to the opening, through which the tip of the syringe may protrude when the syringe is introduced into the housing.

2. A carton according to claim 1 further comprising at least one flap portion which, following introduction of the syringe into the housing, may be moved from a first configuration in which the at least one flap portion does not engage with the syringe into a second configuration in which the at least one flap portion engages with the flange of the syringe to thereby secure the syringe within the housing.

3. A carton according to claim 1 further comprising at least two flap portions which, following introduction of the syringe into the housing may be moved from a first configuration in which the at least two flap portions do not engage with the syringe into a second configuration in which the at least two flap portions engage with the flange of the syringe to thereby secure the syringe within the housing.

4. A carton according to claim 2, wherein the flap portion is dimensioned such that when in the second configuration, an end of the at least one flap portion abuts against the flange of the syringe to thereby secure the syringe within the housing.

5. A carton according to claim 1, wherein the second portion of the chamber comprises indents (e.g., disposed in a position opposite to the opening) which are ergonomically designed (e.g., semi-circular) to engage with the fingers (e.g., fore finger and middle finger) of the user.

6. A carton according to claim 1, wherein the second portion of the chamber comprises plunger indents which are ergonomically designed to allow depression of the plunger of the syringe.

7. A carton according to claim 1, wherein the housing is at least substantially opaque.

8. A carton according to claim 1, wherein the housing is substantially complimentary in shape to the syringe.

9. A carton according to claim 1, wherein the housing is dimensioned such that following introduction of the syringe into the housing, movement of the syringe is substantially prevented.

10. A carton according to claim 1, wherein the carton is formed from the same continuous sheet of material.

11. A carton according to claim 10, wherein the continuous sheet of material is held in shape with an adhesive.

12. A carton according to claim 1, wherein the carton is formed from paperboard.

13. A carton according to claim 1, further including the syringe disposed therein.

14. A carton according to claim 13, wherein the barrel of the syringe is within the first portion of the chamber, the flange of the syringe is within the first portion of the chamber, the tip of the syringe protrudes through the aperture such that the tip is located outside of the housing, and the plunger of the syringe is located outside the opening of the housing.

15. A method of using the carton according to claim 1, comprising obscuring the contents of the barrel of the syringe.

16. The method according to claim 15, wherein the carton obscures the contents of the barrel of the syringe whilst being administered to a patient during clinical trial testing.

17. A blank for forming the carton of claim 1.

18. A blank for a carton comprising:
a single continuous sheet of material, the single sheet comprising:
a first section which forms a back panel of the carton;
a second section which forms a first side panel of the carton;
a third section which forms a front panel of the carton; and
a fourth section which forms a second side panel of the carton;
wherein the first section is joined to the second section along a fold line and/or perforated crease;
wherein the second section is joined to the third section along a fold line and/or perforated crease;
wherein the third section is joined to the fourth section along a fold line and/or perforated crease;
at least one flap extending longitudinally from one or more of the sections in substantially the same direction as at least one of the fold lines and/or perforated creases; and
a tab extending longitudinally from the first section in a direction substantially perpendicular to at least one of the fold lines and/or perforated creases.

19. A blank according to claim 18, further comprising at least two cuts, a first one of the at least two cuts traversing the second section and a portion of the third section in a direction substantially perpendicular to at least one of the fold lines and/or perforated creases and a second one of the at least two cuts traversing the fourth section and a portion of the third section in a direction substantially perpendicular to at least one of the fold lines and/or perforated creases.

20. A product comprising the blank according to claim 18, wherein the blank is folded along the fold lines and/or perforated creases such that the tab contacts an underside of the fourth section and is attached thereto with adhesive to thereby join the tab and the fourth section together.

21. A product comprising the blank according to claim 18, wherein the blank is folded along a further fold line and/or perforated crease such that a portion of the fourth section contacts a portion of the first section and is attached thereto with adhesive to thereby join a portion of the first section to a portion of the fourth section.

22. A method of making a carton comprising folding the blank according to claim 17.

23-24. (canceled)
